(19) United States
(12) Patent Application Publication
(10) Pub. No.: US 2018/0168450 A1
(43) Pub. Date: Jun. 21, 2018

(54) SYSTEMS AND METHODS FOR A VISUALIZATION ALARM

(71) Applicant: General Electric Company, Schenectady, NY (US)

(72) Inventor: Milvi Kristiina Soosalu, Helsinki (FI)

(21) Appl. No.: 15/385,394

(22) Filed: Dec. 20, 2016

(51) Int. Cl. A61B 5/00; A61B 5/0402; A61B 5/0476; A61B 5/0205; A61B 5/1455; A61B 5/021

(52) U.S. Cl. CPC A61B 5/0022; A61B 5/0402; A61B 5/0476; A61B 5/021; A61B 5/0008; A61B 5/14551; A61B 5/0205

(57) ABSTRACT

Systems and methods are provided for an early visualization alarm for a medical monitoring system prior to a critical patient state are provided. The systems and methods receive physiological characteristics of a patient, calculate a physiological value based on the physiological characteristics, determine a presentation style based on a comparison of the physiological value to the first and second predetermined thresholds, and display the physiological value on a display utilizing the presentation style.

### BACKGROUND

The subject matter herein relates generally to systems and methods for an early visualization alarm for a medical monitoring system prior to a critical patient state.

When a patient is admitted into a healthcare facility, the patient is often connected to a plurality of sensors in contact with the patient, such as wearable sensors, cardiac sensor, breathing sensors, and/or the like. The sensors detect and displays physiological information (e.g., cardiac activity, breathing activity, electrocardiography (ECG) data, etc.) of the patient. The physiological information is monitored to determine and alert medical staff when the patient is in an alarm status (e.g., critical condition) by exceeding an alarm limit. When the physiological information value exceeds the alarm limit, the physiological information displayed will be highlighted with an alarm priority background color indicating the alarm status of the patient. The visual presentation of the physiological information alternates by blinking between an alarm background color (red, yellow or blue) and a normal parameter color of the physiological information. Additionally, a sound will alarm in conjunction with the alarm status. Optionally, the clinician may be able to turn off the sound alarm.

However, based on a condition of the patient the conventional parameter monitoring may continually enter the alarm status, which is one annoying element for clinicians with the conventional parameter monitoring. Also the continuous alarming: sounds and bright flashing elements on the screen disturb the patients.

### BRIEF DESCRIPTION

In an embodiment a system (e.g., a medical monitoring system) is provided. The system includes a memory that includes first and second predetermined thresholds. The system also includes a controller circuit communicatively coupled to the memory. The controller circuit is configured to receive physiological characteristics of a patient, calculate a physiological value based on the physiological characteristics, determine a presentation style based on a comparison of the physiological value to the first and second predetermined thresholds, and display the physiological value on a display utilizing the presentation style.

In an embodiment a method (e.g., for a visualization alarm when monitoring a patient) is provided. The method includes receiving physiological characteristics of a patient, calculating a physiological value based on the physiological characteristics, determining a presentation style based on a comparison of the physiological value to the first and second predetermined thresholds, and displaying the physiological value on a display utilizing the presentation style.

In an embodiment a tangible and non-transitory computer readable medium comprising one or more computer software modules is provided. The software modules are configured to direct one or more processors to receive physiological characteristics of a patient, calculate a physiological value based on the physiological characteristics, determine a presentation style based on a comparison of the physiological value to the first and second predetermined thresholds, and display the physiological value on a display utilizing the presentation style.

### BRIEF DESCRIPTION OF THE DRAWINGS

FIG. 1 is a block diagram of an embodiment of a medical monitoring system.

FIG. 2 is a method of an embodiment for a visualization alarm when monitoring a patient.

FIG. 3 is a visualization of a presentation style representative of a default state shown on a display, in accordance with an embodiment.

FIG. 4A is a visualization of a presentation style representative of an early alert state shown on a display, in accordance with an embodiment.

FIG. 4B is a visualization of a presentation style representative of an early alert state shown on a display, in accordance with an embodiment.

FIG. 4C is a visualization of a presentation style representative of an early alert state shown on a display, in accordance with an embodiment.

FIG. 5 is a visualization of a presentation style of critical alert state shown on a display, in accordance with an embodiment

### DETAILED DESCRIPTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments described herein include systems and methods for an early visualization alarm for a medical monitoring system prior to a critical patient state. The medical monitoring system is configured to receive and/or monitor physiological characteristics (e.g., heart rate, breathing rate, oxygen level, cardiac information, blood pressure, bioelectrical information, temperature, acid balance, and/or the like) of a patient. Based on the physiological characteristics, the medical monitoring system is configured to calculate a physiological value based on the physiological characteristics and display the physiological value on the display. The physiological value is displayed utilizing a presentation style. The medical monitoring system is configured to determine the presentation style based on the physiological value with respect to a first and second predetermined threshold.

The second predetermined threshold may represent a critical alert of the patient. For example, the second predetermined threshold may represent the physiological characteristic of the patient that represents a critical condition of the patient requiring clinical attention. The critical condition may correspond to physiological characteristics of the patient that are unstable and/or not within normal limits. For example, prolonged status of the patient in the critical condition may cause damage, irreparable damage, death, and/or the like to the patient.

The first predetermined threshold may represent a warning state preceding the critical condition. For example, the first predetermined threshold may represent the physiological characteristics of the patient that are proximate to the physiological characteristics of the critical condition. The physiological characteristics based on the first predetermined threshold may not be within normal limits but a stable condition of the patient. Additionally or alternatively, the first predetermined threshold may represent conditions of the patient that are not critical. For example, the first predetermined threshold may correspond to a condition of the patient that does not require immediate attention from the clinician.

The physiological value may refer to an indication and/or representation generated by and shown on the display of the medical monitoring system measurement system of the physiological characteristics of the patient. The physiological value includes, but is not limited to, a numerical value, a waveform, a bar graph, a graphical icon, and/or the like.

The presentation style may refer to a distinctive and/or designed appearance of the physiological value shown on the display. The presentation style may include, but is not limited to, a border color, a fill color, a font, a brightness, a color pattern, a boarder thickness, and/or the like. The presentation style is based on the physiological value with respect to the first and second predetermined thresholds. For example, the presentation styles for the physiological value at and/or above the first and/or second predetermined thresholds are configured to adjust an appearance of the physiological value shown on a display at a predetermined cycle rate. During the predetermined cycle rate, the medical monitoring system may be configured to animate and/or adjust the presentation styles to indicate to a clinician the condition of the patient. For example, the medical monitoring system may adjust the color, thickness, color pattern, font, brightness and/or the like of the physiological value to pulsate and/or animate the physiological value shown on the display. The cycle rate may be based on the physiological value relative to the first and second predetermined threshold. For example, the cycle rate when the physiological value is above the second predetermined threshold may be greater and/or larger relative to the cycle rate when the physiological value is below the second predetermined threshold.

A technical effect of the various embodiments include an early warning visualization for clinicians to react to a patient condition prior to a critical condition. A technical effect of the various embodiments include increased patient safety and/or more opportunities for clinicians to provide patient care.

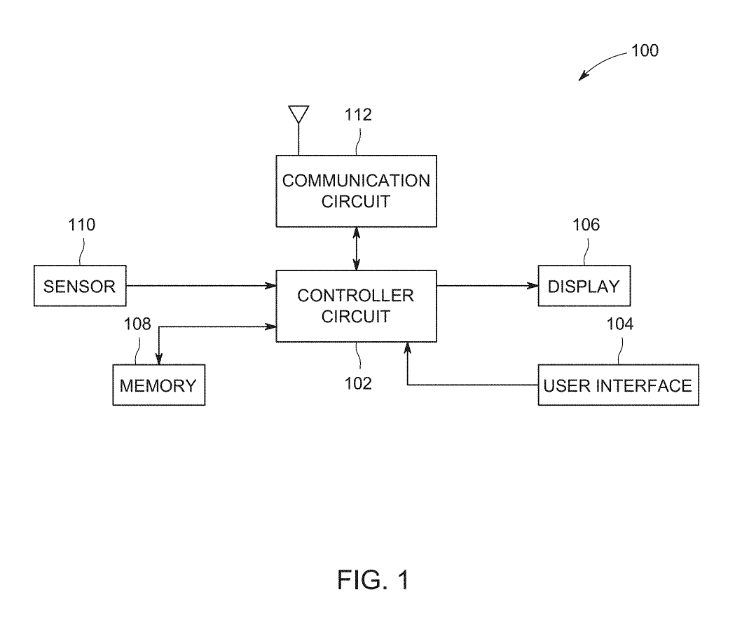

FIG. 1 is a block diagram of an embodiment of a medical monitoring system (MMS) 100. The MMS 100 includes a controller circuit 102, a memory 108, and a display 106. The memory 108 may include the first and second predetermined thresholds utilized by the controller circuit 102. The controller circuit 102 is communicatively coupled to the memory 108. The controller circuit 102 may include and/or represent one or more hardware circuits or circuitry that include, are connected with, or that both include and are connected with one or more processors, controllers, and/or other hardware logic-based devices. Additionally or alternatively, the controller circuit 102 may execute one or more programmed instructions (e.g., software modules) stored on a tangible and non-transitory computer readable medium (e.g., memory 108) to perform one or more operations as described herein.

The controller circuit 102 is operably and/or communicatively coupled to the display 106. The display 106 may include one or more liquid crystal displays (e.g., light emitting diode (LED) backlight), organic light emitting diode (OLED) displays, plasma displays, CRT displays, and/or the like. The display 106 is configured to display one or more physiological values calculated by the controller circuit 102. Additionally or alternatively, the display 106 may display patient information, treatment information, and/or the like.

Optionally, the MMS 100 may include a user interface 104. The user interface 104 may be operatively coupled to the controller circuit 102. The controller circuit 102 may include hardware, firmware, software, or a combination thereof that enables an individual (e.g., an operator) to directly and/or indirectly control operation of the MMS 100 and the various components thereof based on signals received from the user interface 104. The user interface 104 controls operations of the controller circuit 102 and is configured to receive inputs from the user. For example, the user interface 104 may include a keyboard, a mouse, a touchpad, one or more physical buttons, and/or the like.

Optionally, a first and second predetermined threshold may be received by the controller circuit 102. For example, the clinician (e.g., user) utilizing the user interface 104 may define one or more physiological values and/or physiological characteristics representing the first and/or second predetermined thresholds. Based on the signals received from the user interface 104, the controller circuit 102 may store and/or record the first and/or second predetermined thresholds in the memory 108.

Additionally or alternatively, the display 106 may be a touch screen display, which includes at least a portion of the user interface 104 shown as a graphical user interface (GUI). The touch screen display can detect a presence of a touch from the operator on the display 106 and can also identify a location of the touch in the display 106. For example, the user may select one or more user selectable elements shown on the display by touching or making contact with the display 106. The touch may be applied by, for example, at least one of an individual's hand, glove, stylus, or the like.

Optionally, the MMS 100 may include a communication circuit 112. The controller circuit 102 may be operably and/or communicatively coupled to the communication circuit 112. The communication circuit 112 may be controlled by the controller circuit 102 and be configured to establish and/or transmit data along a bi-directional communication link. For example, the bi-directional communication link may be configured to communicatively couple the MMS 100 to a remote system (not shown) such as a nursing station, remote server, broadcast system, and/or the like utilized by one or more clinicians. The bi-directional communication link may be utilized by the MMS 100 to transmit one or more physiological values and/or the presentation styles to the remote system. The communication circuit 112 may include physical layer (PHY) components such as a transceiver, one or more communication ports, a digital signal processor, one or more amplifiers, an antenna, and/or the like for communicatively coupling the MMS 100 to the remote system. The communication circuit 112 may include one or more processors, a central controller circuit (CPU), one or more microprocessors, or any other electronic components capable of processing inputted data according to specific logical instructions.

The bi-directional communication links established by the communication circuit 112 may conform to one or more communication protocols such as an Ethernet Standard, DICOM, USB, one or more wireless standards (e.g., 802.11, Bluetooth, Bluetooth Low Energy, ZigBee), and/or the like. The protocol firmware for the one or more communication protocols may be stored on the memory 108, which is accessible by the communication circuit 112 directly and/or via the controller circuit 102. Additionally or alternatively, the firmware may be stored within an internal memory of the communication circuit 112. The protocol firmware provides the communication protocol syntax for the communication circuit 112 to assemble data packets, establish one or more bi-directional communication links, and/or partition data received from the remote system.

Additionally or alternatively, the MMS 100 may include one or more physiological sensors 110. The one or more physiological sensors 110 are configured to acquire physiological characteristics (e.g., heart rate, blood pressure, respiratory rate, oxygen level, and/or the like) of the patient. For example, the one or more physiological sensor 110 may be configured to acquire cardiac information (e.g., an electrocardiograph (ECG) sensor, electroencephalograph (EEG), a pulse oximeter, blood pressure monitor, and/or the like), respiratory information (e.g., a respiratory sensor), body temperature information (e.g., temperature sensor), and/or the like. The one or more physiological sensors 110 may be operatively and/or communicatively coupled to the controller circuit 102. For example, the controller circuit 102 receives physiological characteristics acquired by the one or more physiological sensors 110. Additionally or alternatively, the one or more physiological sensors 110 may be communicatively coupled to the MMS 100 via corresponding bi-directional communication links with the communication circuit 112.

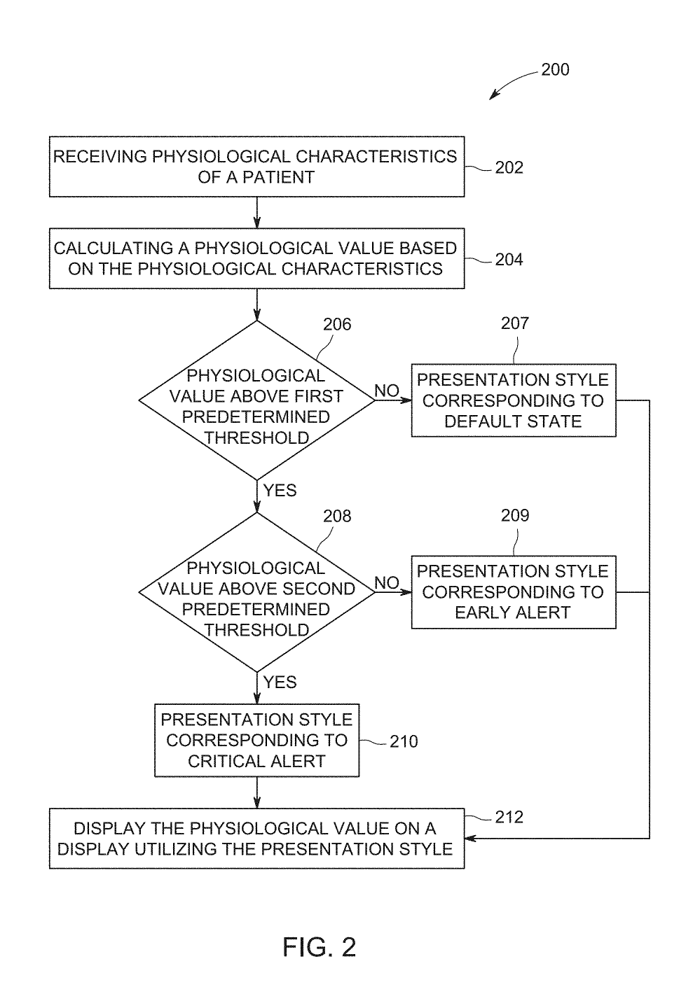

In connection with FIG. 2, the controller circuit 102 is configured to calculate a physiological value based on the physiological characteristics received from the one or more physiological sensors 110. Based on the physiological value, the controller circuit 102 is configured to determine a presentation style for the physiological value to be shown on the display.

FIG. 2 illustrates is a method 200 of an embodiment for a visualization alarm when monitoring a patient. The method 200, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 200 may be used as one or more algorithms to direct hardware to perform one or more operations described herein.

Beginning at 202, the controller circuit 102 is configured to receive the physiological characteristics of a patient. For example, the one or more physiological sensors 110 are positioned in contact with and/or proximate to the patient. Based on the proximity of the one or more physiological sensors 110, the physiological characteristics of the patient are acquires, such as heart rate, blood pressure, respiratory rate, oxygen level, and/or the like. The physiological characteristics may be received by the controller circuit 102 via one or more bi-directional communication links. For example, the physiological sensor 110 may transmit the physiological characteristics within one or more data packets, which are received by the communication circuit 112 via the bi-directional communication link. The communication circuit 112 may partition the physiological characteristics from the one or more data packets via the wired and/or wireless network protocol, which is received by the controller circuit 102. Additionally or alternatively, the one or more physiological sensors 110 may be operably coupled to the controller circuit 102.

At 204, the controller circuit 102 is configured to calculate a physiological value based on the physiological characteristics. The physiological characteristics may be represented as electrical signals (e.g., analog, digital) generated by the one or more physiological sensors 110, which are received by the controller circuit 102. The controller circuit 102 may determine the physiological value based on a configuration of the electrical signals (e.g., frequency, amplitude, peaks, bit sequence, and/or the like). The controller circuit 102 may calculate a numerical value, a waveform, a bar graph, a graphical icon, and/or the like representative of the physiological characteristics received by the controller circuit 102. For example, the physiological characteristics may represent cardiac information of the patient. The electrical signal may include a plurality of peaks representative of a heart beat of the patient. Based on a position and/or timing of the peaks, the controller circuit 102 may calculate a heart rate of the patient as a numerical value. In another example, the physiological characteristics may represent respiratory information of the patient. Based on a frequency and/or timing of the electrical signal, the controller circuit 102 may calculate a lung volume and/or lung compliance of the patient shown as a bar graph.

It may be noted that the controller circuit 102 may calculate a plurality of physiological values based on the physiological characteristics. For example, the controller circuit 102 may calculate physiological values representing cardiac information, respiratory information, and/or the like. Additionally or alternatively, the controller circuit 102 may calculate multiple physiological values based on similar and/or substantially the same physiological characteristics. For example, the physiological characteristics may correspond to cardiac information of the patient. The controller circuit 102 may calculate a physiological value representative of a numerical value, such as a heartbeat rate of the patient, and a physiological waveform, such as an electrocardiography of the patient.

The operations at 206-210 may correspond to the controller circuit 102 configured to determine the presentation style based on a comparison of the physiological value to the first and second predetermined thresholds. The first predetermined threshold may be configured as an early warning preceding the critical condition of the patient. The critical condition may correspond to physiological characteristics of the patient that are unstable and/or not within normal limits. For example, prolonged status of the patient in the critical condition may cause damage, irreparable damage, death, and/or the like to the patient. The first predetermined threshold may represent the physiological characteristics of the patient that are proximate to the physiological characteristics representing a defined critical condition of the patient. For example, the physiological characteristics based on the first predetermined threshold may not be within normal limits but a stable condition of the patient. The second predetermined threshold may be configured to be at the critical alert of the patient. The second predetermined threshold may represent the physiological characteristic of the patient that represents a critical condition of the patient requiring clinical attention.

For example, at 206, the controller circuit 102 is configured to determine whether the physiological value is above the first predetermined threshold. For example, the first and second predetermined thresholds may be based on physiological characteristics corresponding to cardiac information. The first predetermined threshold may represent a heart rate of 87 beats per minute, and the second predetermined threshold may represent a heart rate of 90 beats per minute. The controller circuit 102 is configured to compare the physiological value determined at 204 with the first predetermined threshold.

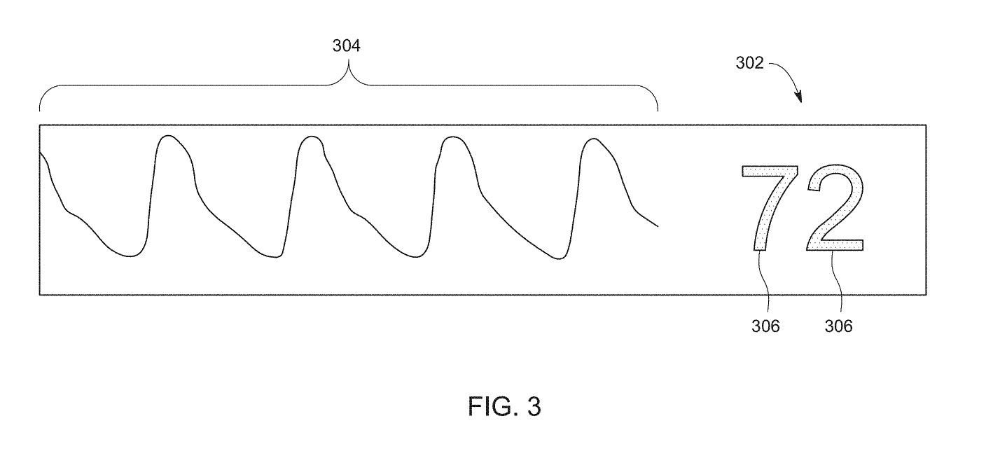

If the controller circuit 102 determines the physiological value is not above the first predetermined threshold, then at 207, the controller circuit 102 is configured to determine the presentation style corresponds to a default state. For example, the controller circuit 102 may determine that the physiological value corresponds to a hear rate of 72 beats per minute. The controller circuit 102 compares the physiological value with the first predetermined threshold, such as 87 beats per minute, and determine the physiological value is below the first predetermined threshold. Based on the physiological value being below the first predetermined threshold, the controller circuit 102 may determine the presentation style corresponds to the default state. The default state may correspond to the presentation style where the appearance of the physiological value is not adjusted. For example, in connection with FIG. 3 the default state may be configured not to be adjusted at a cycle rate.

FIG. 3 is a visualization 300 of a presentation style representative of a default state shown on the display 106, in accordance with an embodiment. For example, the presentation style of the physiological value 302 may have a solid fill color 306. The solid fill color 306 may represent a color of the physiological value 302. Optionally, the physiological value 302 may have a color pattern rather than the solid fill color 306. For example, the presentation style of the visualization 300 may be predetermined and stored in the memory 108. Additionally or alternatively, a physiological waveform 304 may be shown concurrently and/or simultaneously with the physiological value 302. Optionally, the physiological waveform 304 may have the same and/or presentation style (e.g., similar fill color as the solid fill color 306) as the physiological value 302.

Additionally or alternatively, the presentation style representing the default state may be selected by the clinician. For example, the clinician may select the presentation style (e.g., a border color, a fill color, a font, a brightness, a color pattern, a boarder thickness, and/or the like) from a plurality of options utilizing the user interface 104.

If the controller circuit 102 determines the physiological value is above the first predetermine threshold, then at 208, the controller circuit 102 is configured to determine whether the physiological value is above the second predetermined threshold. For example, the controller circuit 102 may determine that the physiological value corresponds to a heart rate of 88 beats per minute. The controller circuit 102 compares the physiological value with the second predetermined threshold, such as 90 beats per minute, and determines the physiological value is below the second predetermined threshold.

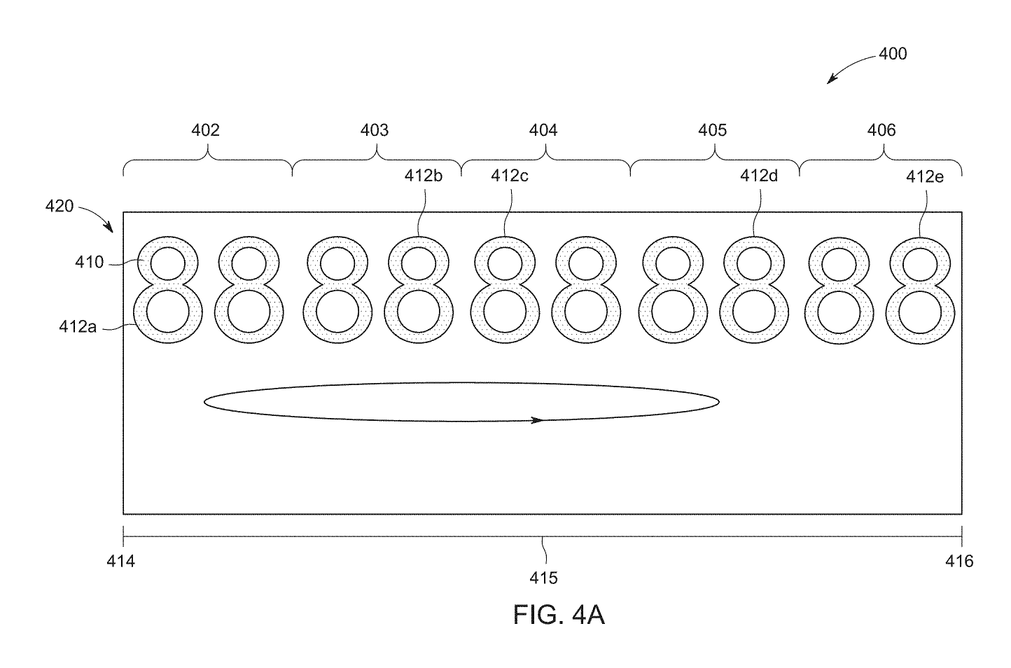

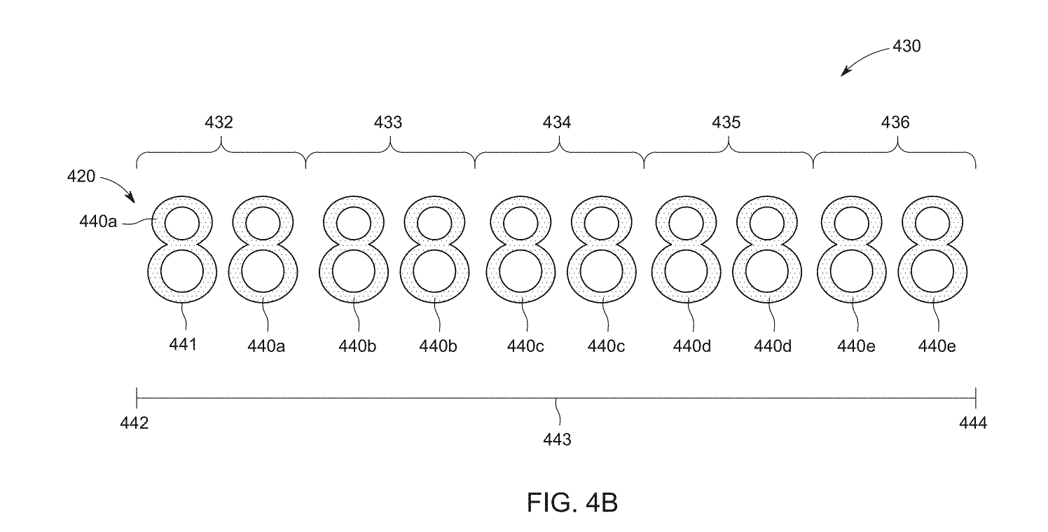

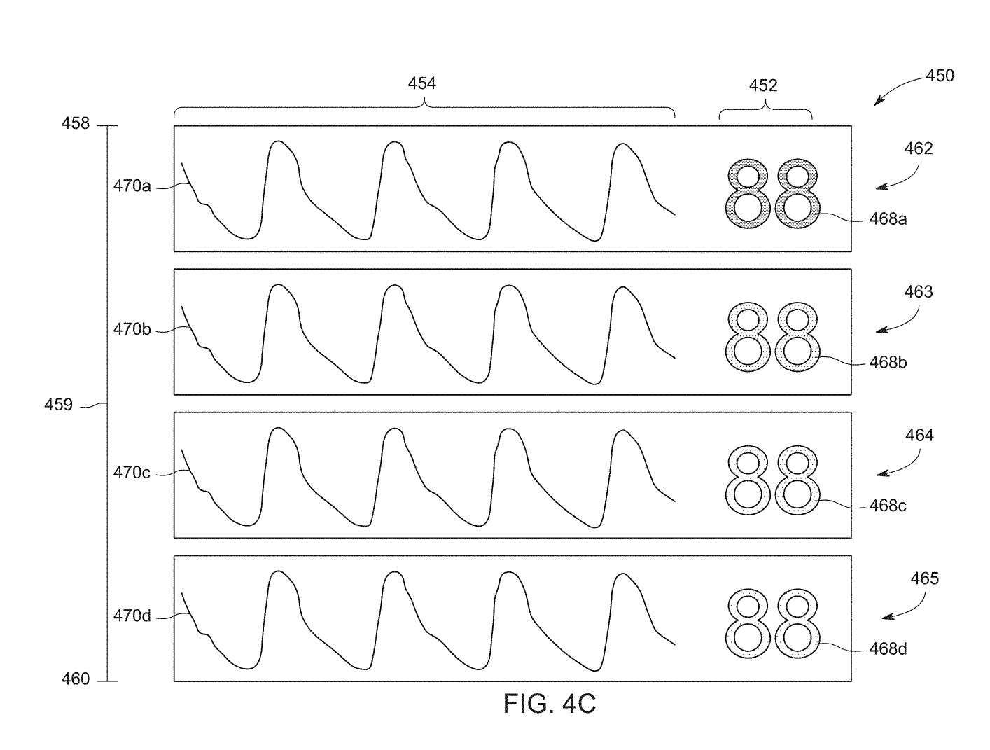

If the controller circuit 102 determines the physiological value is not above the second predetermined threshold, then at 209, the controller circuit 102 is configured to determine the presentation style corresponds to an early alert. For example, in connection with FIGS. 4A-C, the early alert may correspond to a presentation style for the physiological value configured to adjust an appearance of the physiological value shown on the display 106 at a predetermined cycle rate.

FIGS. 4A-C are visualizations 400, 430, 450 of presentation styles 402-406, 432-436, 462-465 representative of early alert states shown on the display 106, in accordance with various embodiments. Optionally, the additional variations of the presentation styles may be predetermined and/or stored in the memory 108. Additionally or alternatively, the additional variations of the presentation styles are selected by the clinician from a plurality of options utilizing the user interface 104. The visualizations 400, 430, 450 are shown adjusting the presentation styles 402-406, 432-436, 462-465 along animation timelines 415, 443, 459 of a physiological value 420 (e.g., shown as a numerical value 88). For example, the visualizations 400, 430, 450 represent animations generated by the controller circuit 102 to be shown on the display 106.

For example, the visualization 400 is shown having a series of presentation styles 402-406. The series of presentation styles 402-406 may represent static images of the animation of the visualization 400 along the animation timeline 415. For example, the controller circuit 102 may synchronously display the sequence of the presentation styles 402-406 at a predetermined cycle rate. Each of the presentation styles 402-406 have a solid fill color 410. The controller circuit 102 may continually transition between the presentation styles 402-406 along the animation timeline 415 by adjusting a thickness of boarders 412a-e of the physiological value 420. The controller circuit 102 continually cycles along the animation timeline 415 between 414 and 415 when the physiological value 420 is above the first predetermined threshold and below the second predetermined threshold. For example, the controller circuit 102 may continually increase the thickness of the boarders 412a-e when traversing along the animation timeline 415 from 414 to 415, and then continually decrease the thickness of the boarders 412a-e when traversing along the animation timeline 415 from 415 to 414. The controller circuit 102 is configured to traverse the animation timeline 415 between 414 and 415 at the predetermined cycle rate. The predetermined cycle rate may represent a time period for the controller circuit 102 to transition from the presentation styles 402 to the presentation style 406. For example, the predetermined cycle rate may represent a frequency (e.g., 1 hertz, 5 hertz, 10 hertz, and/or the like) and/or time period (e.g., 5 seconds, 10 seconds, and/or the like). Additionally or alternatively, the predetermined cycle rate may be selected by the clinician utilizing the user interface 104.

In another example, the visualization 430 is shown having a series of presentation styles 432-436. The series of presentation styles 432-436 may represent static images of the animation of the visualization 430 along the animation timeline 443. For example, the controller circuit 102 may synchronously display the sequence of the presentation styles 432-436 at a predetermined cycle rate. Each of the presentation styles 402-406 have a boundary 441. The controller circuit 102 may continually transition between the presentation styles 432-436 along the animation timeline 443 by adjusting solid fill colors 440a-e of the physiological value 420. The controller circuit 102 continually cycles along the animation timeline 443 between 442 and 444 when the physiological value 420 is above the first predetermined threshold and below the second predetermined threshold. For example, the controller circuit 102 may continually adjust the solid fill colors 440a-e when traversing along the animation timeline 443 from 442 to 444, and then continually adjust the solid fill colors 440a-e when traversing along the animation timeline 443 from 442 to 444.

In another example, the visualization 450 is shown having a series of presentation styles 462-465. The presentation styles 462-465 may include a plurality of physiological values 452, 454. For example, the presentation styles 462-465 each include the physiological value 452 shown as a numerical value, such as 88, and the physiological value 454 shown as a physiological waveform. The physiological values 452 and 454 may be shown concurrently and/or simultaneously with each other on the display 106. Additionally or alternatively, the physiological values 452 and 454 may be shown separately on different displays, overlaid and/or superimposed with each other, and/or the like. The series of presentation styles 462-465 may represent static images of the animation of the visualization 450 along the animation timeline 459. For example, the controller circuit 102 may synchronously display the sequence of the presentation styles 462-465 at one or more predetermined cycle rates. The controller circuit 102 may continually transition between the presentation styles 462-465 along the animation timeline 459 by adjusting solid fill colors 468a-d of the physiological value 452 and waveform colors 470a-d of the physiological value 454. Additionally or alternatively, in an embodiment the presentation styles 462-465 may adjust a color, thickness, brightness, and/or pattern of the physiological value 454 along the animation timeline 459.

The controller circuit 102 continually cycles along the animation timeline 459 between 458 and 460 when the physiological values 452, 454 are above the first predetermined threshold and below the second predetermined threshold. For example, the controller circuit 102 may continually adjust the solid fill colors 468a-d and the waveform colors 470a-d when traversing along the animation timeline 459 from 458 to 460, and then continually adjust the solid fill colors 468a-d and the waveform colors 470a-d when traversing along the animation timeline 459 from 460 to 458. Additionally or alternatively, the controller circuit 102 is configured to traverse the animation timeline 459 at one or more predetermined cycle rates. For example, the controller circuit 102 may adjust the physiological value 454 along the animation timeline 459 at a different rate than the physiological value 452. Additionally or alternatively, the controller circuit 102 is configured to traverse the animation timeline 459 for the physiological values 452, 454 at similar and/or substantially the same predetermined cycle rate.

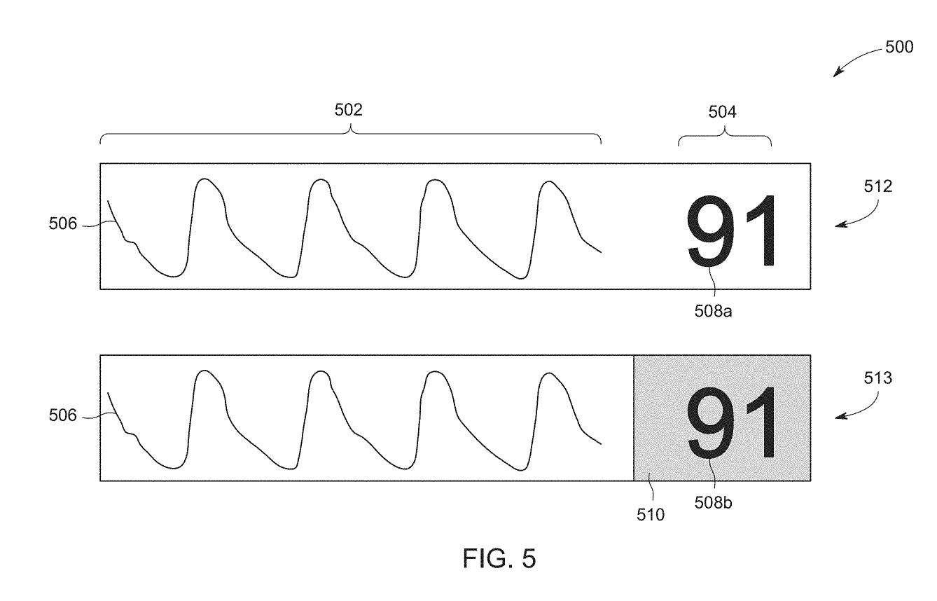

Returning to FIG. 2, if the controller circuit 102 determines the physiological value is above the second predetermined threshold, then at 210, the controller circuit 102 is configured to determine the presentation style corresponds to a critical alert. For example, the controller circuit 102 may determine that the physiological value corresponds to a heart rate of 91 beats per minute. The controller circuit 102 compares the physiological value with the second predetermined threshold, such as 90 beats per minute, and determines the physiological value is above the second predetermined threshold. In connection with FIG. 5, the critical alert state may correspond to presentation styles 512-513 for physiological values 502, 504 configured to adjust an appearance of the physiological value shown on the display 106 at a predetermined cycle rate.

FIG. 5 is a visualization 500 of a presentation style 512, 513 of a critical alert state shown on the display 106, in accordance with an embodiment. Optionally, the additional variations of the presentation styles 512, 513 may be predetermined and/or stored in the memory 108. Additionally or alternatively, the additional variations of the presentation styles 512, 513 are selected by the clinician from a plurality of options utilizing the user interface 104. The visualization 500 is shown adjusting the presentation styles 512-513 the physiological values 502 (e.g., shown as a physiological waveform) and 504 (e.g., shown as a numerical value 91). For example, the visualization 500 represent an animation generated by the controller circuit 102 to be shown on the display 106 by continually switching between the presentation styles 512, 513. A solid fill color 508a-b of the physiological value 504 is adjusted between the presentation styles 512, 513. For example, the solid fill color 508a represents a red color, and the solid fill color 508b represents a white color. Additionally or alternatively, a background 510 is adjusted between the presentation styles 512, 513. For example, the background 510 is adjusted from black at the presentation style 512 to red at the presentation style 513. The visualization 500 includes the physiological value 502 represented as a physiological waveform having a waveform color 506.

The controller circuit 102 may continually transition between the presentation styles 512-513 at a predetermined cycle rate when the physiological values 502 is above the second predetermined threshold. It may be noted that predetermined cycle rate of the visualization 500 may be above and/or greater relative to the predetermined cycle rates of the visualizations 400, 430, 450. For example, the controller circuit 102 may be configured to continually transition between the presentation styles 512-513 at the predetermined cycle rate of 50 hertz, conversely, the controller circuit 102 may be configured to continually traverse along the animation timelines 415, 443, 459 at the predetermined cycle rate of 5 hertz.

At 212, the controller circuit 102 is configured to display the physiological value on the display 106 utilizing the presentation style. For example, the controller circuit 102 is configure to generate the visualizations 300, 400, 430, 450, or 500.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem,” “circuit” or “module” may include a processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term computer,” “subsystem,” “circuit” or “module”. The one or more processors execute a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A medical monitoring system comprising:
a memory that includes first and second predetermined thresholds; and
a controller circuit communicatively coupled to the memory, wherein the controller circuit is configured to:
receive physiological characteristics of a patient;
calculate a physiological value based on the physiological characteristics;
determine a presentation style based on a comparison of the physiological value to the first and second predetermined thresholds; and
display the physiological value on a display utilizing the presentation style.

2. The medical monitoring system of claim 1, wherein the presentation style includes adjusting at least one of a border color, a fill color, a font, a brightness, a color pattern, or boarder thickness over time based on a cycle rate.

3. The medical monitoring system of claim 2, wherein the presentation style of the physiological value above the first predetermined threshold is at a first cycle rate, and the presentation style of the physiological value above the second predetermined threshold is at a second cycle rate, wherein the second cycle rate is greater relative to the first cycle rate.

4. The medical monitoring system of claim 1, wherein the physiological value is a numerical value, a waveform, a bar graph, or a graphical icon.

5. The medical monitoring system of claim 1, wherein the controller circuit is configured to calculate a physiological waveform over time based on the physiological characteristics, and display the physiological waveform concurrently with the physiological value.

6. The medical monitoring system of claim 5, wherein the controller circuit is configured to determine a waveform presentation style based on a comparison of the physiological value to the first and second predetermined thresholds, the waveform presentation style includes adjusting at least one of a color, a thickness, or a brightness of the physiological waveform over time based on third cycle rate.

7. The medical monitoring system of claim 1, further comprising a communication circuit, wherein the controller circuit is configured to transmit the physiological value and the presentation style to a remote system.

8. The medical monitoring system of claim 1, further comprising a physiological sensor configured to acquire the physiological characteristics of the patient, wherein the controller circuit is communicatively coupled to the physiological sensor.

9. The medical monitoring system of claim 1, wherein the second predetermined threshold represents a critical alert of a patient.

10. The medical monitoring system of claim 1, further comprising a user interface communicatively coupled to the controller circuit, wherein the controller circuit is configured to define the first and second predetermined thresholds based on signals received from the user interface.

11. A method of a visualization alarm when monitoring a patient, the method comprising:
receiving physiological characteristics of a patient;
calculating a physiological value based on the physiological characteristics;
determining a presentation style based on a comparison of the physiological value to the first and second predetermined thresholds; and
displaying the physiological value on a display utilizing the presentation style.

12. The method of claim 11, wherein the displaying of the presentation style includes adjusting at least one of a border color, a fill color, a font, a brightness, a color pattern, or boarder thickness over time based on a cycle rate.

13. The method of claim 12, wherein the presentation style of the physiological value above the first predetermined threshold is at a first cycle rate, and the presentation style of the physiological value above the second predetermined threshold is at a second cycle rate, wherein the second cycle rate is greater relative to the first cycle rate.

14. The method of claim 11, wherein the physiological value is a numerical value, a waveform, a bar graph, or a graphical icon.

15. The method of claim 11, further comprising calculating a physiological waveform over time based on the physiological characteristics, and displaying the physiological waveform concurrently with the physiological value.

16. The method of claim 15, further comprising determining a waveform presentation style based on a comparison of the physiological value to the first and second predetermined thresholds, wherein the displaying of the physiological waveform includes adjusting at least one of a color, a thickness, or a brightness of the physiological waveform over time based on third cycle rate of the waveform presentation style.

17. The method of claim 11, further comprising transmitting the physiological value and the presentation style to a remote system.

18. The method of claim 11, wherein the second predetermined threshold represents a critical alert of a patient.

19. The method of claim 11, further comprising receiving the first and second predetermined threshold based on signals received from a user interface.

20. A tangible and non-transitory computer readable medium comprising one or more computer software modules configured to direct one or more processors to:
receive physiological characteristics of a patient;
calculate a physiological value based on the physiological characteristics;
determine a presentation style based on a comparison of the physiological value to the first and second predetermined thresholds; and
display the physiological value on a display utilizing the presentation style.
